(19) United States
(12) Patent Application Publication
(10) Pub. No.: US 2022/0096696 A1
(43) Pub. Date: Mar. 31, 2022

(54) Device For Disinfecting Air By Means Of Combustion

(71) Applicant: ebm-papst Mulfingen GmbH & Co., KG, Mulfingen (DE)

(72) Inventor: Ralph WYSTUP, Kuenzelsau (DE)

(21) Appl. No.: 17/479,771

(22) Filed: Sep. 20, 2021

(30) Foreign Application Priority Data
Sep. 29, 2020 (DE) 10 2020 125 373.7

(51) Int. Cl. A61L 9/03

(52) U.S. Cl. CPC A61L 9/032

(57) ABSTRACT

A device (1) for disinfecting air by combustion has a primary inlet (10) that sucks primary air into a combustion chamber (11) with a burner nozzle (12). The primary air flows along a flow path into a mixing chamber (13) arranged along the flow path after the combustion chamber (11). The primary air (L1) is heated to a first temperature. The mixing chamber (13) has an outlet (14) and at least one secondary inlet (15). Secondary air can be sucked (L2) into the mixing chamber (13) through the inlet (15). The mixing chamber (13) produces a mixed air (L3) of a third temperature of at least 100° C. from a mixing of the primary air (L1) and the secondary air (L2). The mixed air (L3) is held in the mixing chamber (13) for a predetermined residence time, and discharges the mixed air (L3) along the flow path through the outlet (14).

### CROSS-REFERENCE TO RELATED APPLICATION

This application claims priority to German Patent Application No. 10 2020 125 373.7 filed Sep. 29, 2020. The entire disclosure of the above application is incorporated herein by reference.

### FIELD

The disclosure relates to a device for disinfecting air by combustion.

### BACKGROUND

Some bacteria and viruses, such as also Covid-19, can be present in air as an aerosol or adhering to or enclosed by water droplets. Accordingly, it is desirable to be able to purify large amounts of air, in particular, when using air conditioners or generally in closed spaces, by eliminating these bacteria and viruses.

Different approaches for disinfecting air are known in the prior art. However, they are usually not suitable to constantly disinfect large amounts of air, that is to say to render harmless the bacteria or viruses present in the air.

For example, it is already known to disinfect the air by UVC light. Moreover, it is also known, in principle, to disinfect air by microwave radiation or heat. The devices provided for this purpose in the prior art devices allow mostly just purifying comparatively small amounts of air.

The disclosure is therefore based on the object to overcome the abovementioned disadvantages. The disclosures provides a device and an associated method where large amounts of air can be disinfected or purified effectively and efficiently.

### SUMMARY

This object is achieved by a device for disinfecting air by combustion. The device includes a primary inlet enabling a primary air to be sucked into a combustion chamber with a burner nozzle arranged, or in front of, the chamber. The primary air can flow through the combustion chamber along a flow path. A mixing chamber is arranged along the flow path after the combustion chamber. A mixing ratio of a fuel inflowing through the burner nozzle and the primary air is chosen such that the fuel inflowing through the burner nozzle burns completely in the combustion chamber. The primary air can be heated to a first temperature. The mixing chamber has an outlet and at least one secondary inlet where secondary air can be sucked into the mixing chamber. The mixing chamber produces a mixed air of a third temperature of at least 100° C., from mixing the primary air at the first temperature and the secondary air at a second temperature. The mixed air is held for a predetermined residence time at the third temperature. The mixed air is discharged along the flow path through the outlet.

According to the disclosure, a device is proposed for disinfecting air by combustion and preferably gas combustion or by heat generated during combustion. For this purpose, the device comprises a primary inlet, through which a primary air, for example from the surroundings of the device or from a room to be ventilated, can be sucked into the device. Furthermore, a combustion chamber is provided that is fluidly connected with the primary inlet where primary air can flow along a flow path. The combustion chamber is provided with a burner nozzle arranged or in front of it. Furthermore, the device comprises a mixing chamber arranged along the flow path after the combustion chamber. Thus, it is fluidly connected with the combustion chamber. A mixing ratio of a fuel inflowing through the burner nozzle, which can be, for example, oil, preferably gas or other, in particular, fossil energy sources. The primary air is chosen such that the fuel inflowing through the burner nozzle burns completely in the combustion chamber, or in a combustion zone that is located in the combustion chamber. The primary air can be heated to a first temperature, that is preferably above 1000° C., particularly in the case of gas combustion. The mixing chamber comprises an outlet that leads, for example, to a space to be heated or ventilated or to a downstream system for further treatment of the air. At least one secondary inlet is included where secondary air, for example from the surroundings of the device or a space to be ventilated, can be sucked into the mixing chamber. The mixing chamber is designed to produce mixed air of a third temperature of at least 100° C. Preferably, at a third temperature in the range between 150° C. and 250° C. Particularly preferably, at a third temperature of about 200° C. from mixing the primary air at the first temperature and the secondary air at a second temperature. Here, the secondary air can be at ambient temperature. To produce the correct mixing ratio, the volume flow of the primary air and secondary air flowing into the mixing chamber can be measured. Also, the temperature of the inflowing primary air and secondary air can be captured. Furthermore, the mixing chamber is designed, for example, with appropriate insulation and a corresponding dimensioning of the mixing chamber that is essential for the length of the flow path of the mixed air through the mixing chamber. Thus, this holds the mixed air for a predetermined residence time at the third temperature. The mixed air is then discharged along the flow path through the outlet. For the mixed air to reside in the mixing chamber for the predetermined residence time, the mixing chamber can comprise corresponding flow guiding elements. Alternatively, the portion of the mixing chamber where the mixed air, at the third temperature, is held for the predetermined residence time or where the mixed air flows towards the outlet, can also be referred to as the disinfection section of the mixing chamber.

As previously explained, viruses, such as those of the type Covid-19, are mostly dissolved in aerosols or droplets, which are exhaled by humans, for example. Many viruses and bacteria denature when heated above 100° C., that is to say, they are destroyed. This is best done with a flame and heated air. For this purpose, primary air is sucked in and preferably burned with a fuel such as, for example, propane gas. Here, the primary air is heated to over 1000° C., leading to a comparatively safe disinfection of the primary air, however, constituting an unnecessarily high temperature. For the purpose of increasing the air volume flow that can be disinfected and at the same time also reducing the amount of CO₂ generated by the combustion, the hot primary air is mixed with the cold secondary air. Thus, the primary air is cooled and the secondary air is heated. The resulting mixed air is at the third temperature of preferably about 200° C. Furthermore, the mixing ratio of the primary air and the secondary air is preferably set so that the proportion of CO₂ in the mixed air is about 800 ppm (parts per million).

By controlling the supply air flow and the exhaust air flow of air from the mixing chamber, that is to say by controlling the volume flow of the primary air and the volume flow of the secondary air into the mixing chamber and the volume flow of the mixed air from the mixing chamber, the residence time or the residence time of the mixed air at the third temperature in the mixing chamber can be set so that safe decontamination takes place at this temperature.

A space can be supplied with the disinfected mixed air flowing out of the mixing chamber through the outlet via a correspondingly insulated pipe system. If cold air is needed instead of the mixed air at, for example, about 200° C., the mixed air flowing out of the outlet of the mixing chamber can be routed into a downstream system. A downstream system, for example, an air conditioning system or a system exchanging and using the heat, so that in this system, the mixed air will be cooled down to a predetermined temperature. For example, a downstream heat exchanger can be provided that extracts heat from the mixed air and uses it for heating a building, heating water or even for preheating the primary and/or secondary air.

In addition, the correct functioning of the device according to the disclosure can be ensured by using sensors. The sensors constantly monitor the operating parameters of the components of the device or parameters of the primary, secondary and mixed air.

In winter, in a time of heightened infection risk, the described device is particularly efficient as a result of the additional heating effect. The mixed air can be cooled to a room temperature because the thermal energy contained in the mixed air after flowing out of the device can be transferred to a heating system by a heat exchanger.

Through the transparent principle of flame disinfection, high reliability of the device and of a system based on the device is apparent.

The mixed air flowing out through the outlet can be used as disinfected air following cooling, the air, however, contains CO₂. A CO₂ separator or filter can be provided as an additional system for post-treatment of the mixed air. Here, the CO₂ is removed from the mixed air by filtration.

According to an advantageous variant of the disclosure, a primary air fan is arranged along the flow path before or after the combustion chamber. The primary air fan is designed to suck the primary air through the primary inlet and to convey or blow it into the combustion chamber. For this purpose, the primary air fan can also be located between the burner nozzle and a combustion zone arranged in the combustion chamber. The actual combustion takes place in the combustion zone. Thus, the primary air fan is not only used to convey primary air but at the same time also to mix the primary air with the fuel. Alternatively, the primary air fan can be arranged in the combustion chamber and before or after a combustion zone arranged in the combustion chamber where the actual combustion takes place.

Along the flow path of the primary air from the primary inlet to the mixing chamber, the combustion chamber can be delimited or divided by a flow-through protective grid towards the burner nozzle and/or towards the mixing chamber. When dividing the combustion chamber by protective grid, the burner nozzle and the primary air fan air are arranged along the flow path of the primary air before the protective grid. The combustion zone is arranged after the protective grid. The protective grid serves to prevent backfiring of flames or combustion.

In the actual combustion zone in the combustion chamber, a temperature sensor may also be provided. Thus, monitoring occurs of the combustion temperature or the temperature of the primary air during inflow into the mixing chamber.

Alternatively, the combustion zone can also be arranged directly at the burner nozzle. In this case, it preferably comprises an ignition device and at least one sensor for flame monitoring. The primary air fan is preferably arranged before the burner nozzle in the direction of flow of the primary air. Thus, the primary air fan is not continuously exposed to a thermal load by the heated air that is preferably about 1000° C.

In order to be able to control the air volume flow of the secondary air flowing into the mixing chamber, a secondary fan is utilized. This occurs against any air pressure in the mixing chamber that is possibly elevated with respect to the surroundings. The secondary air fan is associated with the at least one secondary inlet in each case. The secondary air fan is designed to suck the secondary air through the respective secondary inlet and to blow it into the mixing chamber.

According to a further advantageous configuration, a mixing fan is arranged in the mixing chamber to mix the primary air with the secondary air. This prepares the mixed air to be as uniform as possible resulting in the primary air and the seconding air in the mixed air with a temperature distribution as homogenous as possible.

In order to be able to check whether the mixed air flowing out of the mixing chamber through the outlet was indeed exposed sufficiently long to a sufficiently high temperature, to assume the substantially complete disinfection, a measuring device or at least a sensor for detecting oxygen content, temperature, pressure, rate and/or fuel content is provided. This captures one or more of the abovementioned properties of the mixed air. It can be arranged in the mixing chamber and/or along the flow path at or in the outlet.

Moreover, an advantageous further development of the device provides that, in or at the outlet, a throttle device is provided. It throttles the volume flow of the mixed air along the flow path through the outlet. Thus, the residence time of the mixed air in the mixing chamber and the volume flow of the mixed air through the outlet from the mixing chamber can be controlled.

In addition, another aspect of the disclosure relates to a method for air disinfection by combustion with a device according to the disclosure. In the method, the primary air is sucked through the primary inlet and mixed with the fuel supplied through the burner nozzle to form a fuel air mixture. The mixing ratio of the primary air and the fuel is chosen such that the fuel is burned completely in the combustion chamber. The primary air is heated to the first temperature during combustion. Then, the primary air, heated to the first temperature, is routed into the mixing chamber. In the mixing chamber, it is mixed with the secondary air at the second temperature. The secondary air is sucked through the at least one secondary inlet to form a mixed air at the third temperature of at least 100° C. This mixed air remains in the mixing chamber for a predetermined residence time. The mixing chamber can comprise a maze-like flow channel, where the mixed air must flow on its way to the outlet of the mixing chamber to maintain residence time. Subsequently, the mixed air can flow out of the outlet of the mixing chamber so that viruses and bacteria present in the mixed air are killed by the application of the third temperature for the predetermined residence time. Thus, the mixed air flowing out of the outlet is disinfected and at least a portion of the viruses and bacteria contained therein have been killed.

The generated mixed air is preferably intended to be used as ambient air. Thus, a mixing ratio of primary air and secondary air is chosen such that the resulting mixed air can be used directly as ambient air. Thus, the air has a sufficiently low proportion of combustion products, such as, for example CO₂.

A method according to the disclosure can be executed, for example, with a hot air generator with a heating power of 10 kW and a volume flow of, for example, 350 m³/h. The air generator can be part of the device according to the disclosure and to this end, comprises at least the combustion chamber and the burner nozzle. The primary air heated by the hot air generator is subsequently mixed with the secondary air in the mixing chamber.

The feasibility of the device or the method implemented with it results from the following, exemplary calculation:

I. Primary Air

The exemplary hot air generator requires 728 g/h propane (C₃H₈) as fuel to heat an air volume flow of 350 m³/h.

$$Equation 1$$

$$( EQ 1 \text{:} combustion reaction of propane ) \text{:}$$

$${C}_{3} {H}_{8} + 5 {O}_{2} \to 3 {CO}_{2} + 4 {H}_{2} O$$

$$Moles \text{:} 3 •12 + 1 •85•32 g \text{/} mol 3 ( 12 + 32 ) + 4 ( 2 + 16 ) g \text{/} mol \\ 44 + 160 g \text{/} mol 132 + 72 g \text{/} mol$$

Per Hour:

350 m³ of Air Contain:

78% N₂ (28 g/mol); 21% O₂; ˜1% noble gases; 0.04% CO₂

$$At standard pressure \to 22.4 liters 1 mole$$

$$350 {m}^{3} air ( \underline{\underline{primary air}} ) \to 78 \% = 273 , 000 l \to 341.25 kg$$

$$25 \% = 73 , 500 l \to 105 kg$$

$$0.04 \% = 140 l ⇒ 0.275 kg$$

Burned:

728 g of C₃H₈→EQ1·728/44 mol=EQ1·16.55 mol
÷O₂ consumption=16.55 mol·160 g/mol=2.65 kg
→CO₂ generated=16.55 mol·132 g/mol=2.185 kg

II. Ratio of Primary Air and Secondary Air to Produce the Mixed Air

21% O₂ [symbol] 105 kg→105 kg−2.65 kg=102.35 kg [symbol] 20.47%
0.04% CO₂ [symbol] 0.275 kg→0.275 kg+2.185 kg=2.46 kg [symbol] 0.36% [symbol]
3600 ppm

According to Pettenkofer max. 1000 ppm better 800 ppm CO₂ in ambient air for a high ambient air quality.

→3600/800 ppm=4.5→4.5·primary air=4.5·350 m³/h=1575 m³/h with 728 g of gas

Therefore, to generate a mixed air having a high ambient air quality, the primary air must be mixed with 4.5-times secondary air in order to achieve a sufficiently low level of CO₂ in the mixed air.

III. Temperature of the Mixed Air (Third Temperature)

Temperature of the propane gas flame or the air heated with it: 1925° C.;

As a result of the assumed inhomogeneous heating of the primary air, an average of 1000° C. is assumed as the first temperature.

For secondary air, 20° C. are assumed as a second temperature.

Richmann's rule of mixing for determining the third temperature: Tₘ=(m₁·T₁+m₂·T₂)/(m₁+m₂)=(1·1000+4.5·20)/(1+4.5) ° C.=198° C.

As the mixing temperature (third temperature), 198° C. is obtained, so that the mixing temperature is sufficiently high, to eliminate viruses such as, for example, Covid-19 from the mixed air or the primary and secondary air, to disinfect it.

The above-disclosed features can be combined in any way to the extent technically possible and not contradictory.

Further areas of applicability will become apparent from the description provided herein. The description and specific examples in this summary are intended for purposes of illustration only and are not intended to limit the scope of the present disclosure.

Other advantageous further developments of the disclosure are identified in the dependent claims or presented below together with the description of the preferred embodiment of the disclosure with reference to the FIGURE.

### DRAWINGS

Other advantageous further developments of the disclosure are identified in the dependent claims or presented below together with the description of the preferred embodiment of the disclosure with reference to the FIGURE.

FIG. 1 is a schematic view of a device according to an advantageous embodiment.

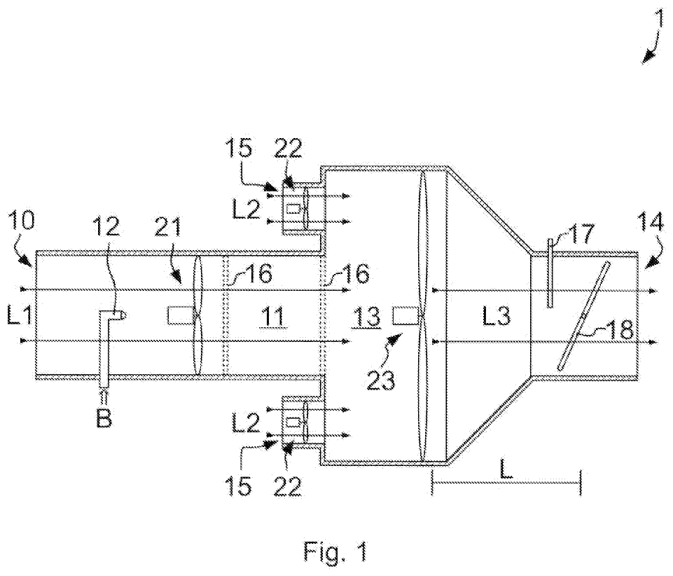

### DETAILED DESCRIPTION

The FIGURE shown schematically is an example and shows a device 1 for disinfecting air by means of gas combustion in a longitudinal section.

### DETAILED DESCRIPTION

The FIGURE shown schematically is an example and shows a device 1 for disinfecting air by gas combustion in a longitudinal section.

For this purpose, primary air L1 is sucked by a primary air fan 21 through a primary inlet 10 into a combustion chamber 11. Here, the primary air L1 is mixed with a fuel B, in this case a gas as fuel B, through burner nozzle 12. The burner nozzle 12 is configured as a gas burner nozzle. In order to delimit the combustion chamber 11 from surrounding areas, or to divide the combustion chamber 11 into subsections, two protective grids 16 are provided. The grids 16 are spaced apart along the direction of flow of primary air L1. They are arranged after burner nozzle 12 and primary air fan 21. This delimits a combustion zone within combustion chamber 11.

By combustion of the fuel-air mixture, primary air L1 is heated to above 1000° C., wherein the fuel is completely burned. In the combustion however, combustion products can form, such as, for example, CO₂.

The heated primary air L1 flows from combustion chamber 11 into mixing chamber 13. Here, primary air L1 is mixed with a secondary air L2 that is sucked in through, in this case, two secondary inlets 15 by a secondary air fan 22 in each inlet. In this case, secondary air fans 22 and primary air fan 21 suck the respective air from the same surroundings or the same room.

Although the secondary air L2 is not involved in the combustion in the combustion chamber 11, it may be heated to a temperature sufficient to kill viruses or bacteria also present in the secondary air L2. The hot exhaust air of the combustion of the primary air L1 flowing into the mixing chamber 13 kills the viruses or bacteria.

A mixing fan 23 is arranged in mixing chamber 13. The mixing fan 23 mixes the primary air L1 and the secondary air L2. Thus, the resulting air mixture or the resulting mixed air L3 has a third temperature as homogenous as possible that corresponds to at least 100° C. and preferably about 200° C.

Mixed air L3 is held at the third temperature in the section following the mixing fan 23 along the flow path of the air through mixing chamber 13. Thus, mixed air L3 along this section and during the residence time of mixed air L3 in this section is exposed to the third temperature for sufficient time to kill the viruses and bacteria still contained in mixed air L3. This section can also be referred to as a disinfection section of mixing chamber 13.

To control the residence time in mixing chamber 13, length L of mixing chamber 13, which is relevant for the length of the flow path of mixed air L3, can be adjusted to a maximum volume flow to be conveyed and a maximum flow rate. Thus, mixed air L3, even at a maximum discharge from outlet 14, is exposed sufficiently long to the third temperature.

To check whether the mixed air has the appropriate parameters, and also for controlling the components such as burner nozzle 12 and fans 21, 22, 23, a measuring device 17 is provided in the area of outlet 14. The measuring device 17 captures the parameters of mixed air 13 relevant for checking and controlling. For this purpose, measuring device 17 may include several sensors.

Furthermore, a throttle device 18 is provided at a transition area from mixing chamber 13 to outlet 14. In the present case, it is designed as a throttle valve. The volume flow of mixed air L3, from mixing chamber 13, and, together with fans 21, 22, 23, the air pressure in mixing chamber 13 can be controlled by throttle device 18.

Following outlet 14, further systems, such as, for example, a heat exchanger, can be provided to extract heat not needed for further use from mixed air L3, and reuse the heat.

Heat extracted from mixed air L3 by a heat exchanger can be used for preheating the primary air and/or the secondary air. Thus, an even larger air volume flow can be brought to the third temperature and thus can be disinfected.

The disclosure in its implementation is not limited to the preferred exemplary embodiments specified above. Rather, a number of variants is conceivable that makes use of the illustrated solution even in case of principally different implementations.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A device for disinfecting air by combustion, comprising:
a primary inlet where a primary air can be sucked in, a combustion chamber with a burner nozzle arranged in or in front of the chamber, primary air can flow through the combustion chamber along a flow path, and a mixing chamber is arranged along the flow path after the combustion chamber;
a mixing ratio of a fuel inflowing through the burner nozzle and the primary air is chosen such that the fuel inflowing through the burner nozzle burns completely in the combustion chamber, and the primary air can be heated to a first temperature;
the mixing chamber has an outlet and at least one secondary inlet where a secondary air can be sucked into the mixing chamber;
the mixing chamber produces a mixed air of a third temperature of at least 100° C. from mixing the primary air at the first temperature and the secondary air at a second temperature, the mixed air is held for a predetermined residence time at the third temperature, and the mixed air is discharged along the flow path through the outlet.

2. The device according to claim 1, wherein a primary air fan is arranged along the flow path before or after the combustion chamber, the primary air fan sucks the primary air through the primary inlet and conveys it into the combustion chamber.

3. The device according to claim 1, wherein the combustion chamber is delimited by a flow-through protective grid along the flow path towards the burner nozzle and/or towards the mixing chamber.

4. The device according to claim 1, wherein a secondary air fan is associated with the at least one secondary inlet, the secondary air fan is designed to suck the secondary air through the respective secondary inlet and to blow it into the mixing chamber.

5. The device according to claim 1, wherein a mixing fan is arranged in the mixing chamber for mixing the primary air with the secondary air and for preparing the mixed air.

6. The device according to claim 1, wherein a measuring device for detecting oxygen content, temperature, pressure, rate and/or fuel content of the mixed air is arranged in the mixing chamber and/or along the flow path at or in the outlet.

7. The device according to claim 1, wherein a throttle device for throttling the volume flow of the mixed air along the flow path through the outlet is included in or at the outlet where the residence time of the mixed air in the mixing chamber can be controlled.

8. A method for air disinfection by combustion with a device according to claim 1 comprising:
sucking the primary air through the primary inlet and mixing the primary air with the fuel supplied through the burner nozzle;
choosing a mixing ratio of the primary air and the fuel such that the fuel is burned completely in the combustion chamber, and heating the primary air to the first temperature during combustion;
routing the primary air heated to the first temperature into the mixing chamber and, in the mixing chamber, mixing the primary air with the secondary air at the second temperature to form a mixed air at the third temperature of at least 100° C., maintaining the mixing air in the mixing chamber for a predetermined residence time and subsequently flowing out of the outlet of the mixing chamber, killing viruses and bacteria present in the mixed air by the application of the third temperature for the predetermined residence time and disinfecting the mixed air flowing out of the outlet.
